SEQUENCE LISTING (24 sequences)

SEQ ID NO: 1 - DNA, 783 bp, Craterostigma plantagineum
  CDS (1)..(783)
atggcgtttctgggccgcattattatttttgcgacctttctggcgattaccagcagcagccattttgcgcgcgcgtattatggcggcgatggcggctggaccgatgcgcatgcgaccttttatggcggcagcgatgcgagcggcaccatgggcggcgcgtgcggctatggcaacctgtatagccagggctatggcaccaacaccgcggcgctgagcaccgcgctgtttaacaacggcctgagctgcggcagctgctttgaaattaaatgcgcgagcagcattagcggcggcggcaaatggtgcctgccgggcggcagcattaccgtgaccgcgaccaacttttgcccgccgaacaacgcgctgccgaacaacgcgggcggctggtgcaacccgccgctgcagcattttgatctgagccagccggtgtttcagcatattgcgcagtatcgcgcgggcattgtgccggtgagctatcgccgcgtgccgtgccgccgccgcggcggcattcgctttaccattaacggccatagctattttaacctggtgctgattaccaacgtgggcggcgcgggcgatgtgcatgcggtgagcattaaaggcgcgaccaccgattggcagccgatgagccgcaactggggccagaactggcagagcaacgcgaacccgaacggccagcgcctgagctttaaagtgaccaccagcgatggccgcaccctggtgagcaacaacgtggcgccgccgaactggagctttggccagacctttgcgggcgcgcagtttaac

SEQ ID NO: 2 - protein, 261 aa, Craterostigma plantagineum
MAFLGRIIIFATFLAITSSSHFARAYYGGDGGWTDAHATFYGGSDASGTMGGACGYGNLYSQGYGTNTAALSTALFNNGLSCGSCFEIKCASSISGGGKWCLPGGSITVTATNFCPPNNALPNNAGGWCNPPLQHFDLSQPVFQHIAQYRAGIVPVSYRRVPCRRRGGIRFTINGHSYFNLVLITNVGGAGDVHAVSIKGATTDWQPMSRNWGQNWQSNANPNGQRLSFKVTTSDGRTLVSNNVAPPNWSFGQTFAGAQFN

SEQ ID NO: 3 - DNA, 657 bp, Craterostigma plantagineum
  CDS (1)..(657)
gcgaccttttatggcggcggcgatgcgagcggcaccatgggcggcgcgtgcggctatggcaacctgtatagcaccggctatggcaccaacaccgcggcgctgagcaccgcgctgtttaacaacggcctgacctgcggcgcgtgctatgaactgacctgcaacaacgatccgcgcggctggtgcctgagcggcaccattatggtgaccgcgaccaacttttgcccgccgaacccgagcctgccgaacgataacggcggctggtgcaacccgccgcgccagcattttgatctggcggaaccggcgtttctgcagattgcgcagtataaagcgggcattgtgccggtgaactatcgccgcgtgccgtgccagaaaaaaggcggcattcgctttaccattaacggccatagcttttttaacctggtgctggtgaccaacgtgggcggcgtgggcgatgtgcatagcgtgagcattaaaggcagcaacggcggctggcagccgctgagccgcaactggggccagaactggcagagcaacagctatctgaacggccagagcctgagctttcaggtgaccaccagcgatggccgcaccgtgaccagctatgatgtggcgccgcgcggctggcagtttggccagacctttgaaggcggccagttt

SEQ ID NO: 4 - protein, 219 aa, Craterostigma plantagineum
ATFYGGGDASGTMGGACGYGNLYSTGYGTNTAALSTALFNNGLTCGACYELTCNNDPRGWCLSGTIMVTATNFCPPNPSLPNDNGGWCNPPRQHFDLAEPAFLQIAQYKAGIVPVNYRRVPCQKKGGIRFTINGHSFFNLVLVTNVGGVGDVHSVSIKGSNGGWQPLSRNWGQNWQSNSYLNGQSLSFQVTTSDGRTVTSYDVAPRGWQFGQTFEGGQF

SEQ ID NO: 5 - DNA, 672 bp, Craterostigma plantagineum
  CDS (1)..(672)
tgccatgcgaccttttatggcggcagcgatgcgagcggcaccatgggcggcgcgtgcggctatggcaacctgtatagccagggctatggcaccaacaccgcggcgctgagcaccaccctgtttaacaacggcctggcgtgcggcagctgctatcaggtgcgctgcgaaggcggcccgaaatggtgcgtgcgcggcggcgatcgcattattaccgtgaccgcgaccaacttttgcccgccgaactatgcgctggcgaacgataacggcggctggtgcaacccgccgcgccagcattttgatatggcgcagccggcgtttgtgcgcattgcgcattatcgcgcgggcattgtgccgattagctatcgccgcgtgagctgcgtgaaaaaaggcggcattcgcctgaccattaacggccatagctattttaacctggtgctggtgagcaacgtgggcggcagcggcgatgtgcatgcggtgtggattaaaggcagcggcggcggcccgtggcaggcgatgacccgcaactggggccagaactggcagagcaacagctatctggatggccagagcctgagctttattgtgcgcgcgggcgatggccgcaccgtgaccgcgaacgabattgtgccgcgcggctggcagtttggccagacctttgaaggcccgcagttt

SEQ ID NO: 6 - protein, 224 aa, Craterostigma plantagineum
  misc_feature (207)..(207): The ′Xaa′ at location 207 stands for Glu, or Asp.
CHATFYGGSDASGTMGGACGYGNLYSQGYGTNTAALSTTLFNNGLACGSCYQVRCEGGPKWCVRGGDRIITVTATNFCPPNYALANDNGGWCNPPRQHFDMAQPAFVRIAHYRAGIVPISYRRVSCVKKGGIRLTINGHSYFNLVLVSNVGGSGDVHAVWIKGSGGGPWQAMTRNWGQNWQSNSYLDGQSLSFIVRAGDGRTVTANXIVPRGWQFGQTFEGPQF

SEQ ID NO: 7 - protein, 8 aa, Craterostigma plantagineum
ASSISGGG

SEQ ID NO: 8 - DNA, 22 bp, Artificial Sequence
  degenerative primer
gsncaygcnacnttytayggng

SEQ ID NO: 9 - DNA, 21 bp, Craterostigma plantagineum
  misc_feature (1)..(1): y is Pyrimidine
ytgccarttytgnccccartt

SEQ ID NO: 10 - DNA, 39 bp, Craterostigma plantagineum
gaccacgcgtatcgatgtcgacttttttttttttttttv

SEQ ID NO: 11 - DNA, 22 bp, Craterostigma plantagineum
gtcacagcctgttttccaacac

SEQ ID NO: 12 - DNA, 18 bp, Craterostigma plantagineum
actaacgtgggcggtgct

SEQ ID NO: 13 - DNA, 25 bp, Craterostigma plantagineum
gcctcctcttctacggcatggtacc

SEQ ID NO: 14 - DNA, 39 bp, Craterostigma plantagineum
aagcagtggtatcaacgcagagtggccattatggccggg

SEQ ID NO: 15 - DNA, 23 bp, Craterostigma plantagineum
aagcagtggtatcaacgcagagt

SEQ ID NO: 16 - DNA, 26 bp, Craterostigma plantagineum
ctgtaagaaacgggcacaatcccagc

SEQ ID NO: 17 - DNA, 24 bp, Craterostigma plantagineum
gctcagtatacagctgggattgtg

SEQ ID NO: 18 - DNA, 27 bp, Craterostigma plantagineum
ttgaagtaagagtgtccgtttattgtg

SEQ ID NO: 19 - DNA, 21 bp, Craterostigma plantagineum
gtgcctctcgggaaccataat

SEQ ID NO: 20 - DNA, 23 bp, Craterostigma plantagineum
ttgtactgcgctatctgcaagaa

SEQ ID NO: 21 - DNA, 21 bp, Craterostigma plantagineum
cgctgagtacaacgctgttca

SEQ ID NO: 22 - DNA, 25 bp, Craterostigma plantagineum
gtaattgggaggacagaaatttgtg

SEQ ID NO: 23 - DNA, 23 bp, Craterostigma plantagineum
catctgggttaagaacggaaaca

SEQ ID NO: 24 - DNA, 23 bp, Craterostigma plantagineum
caaaaccgatcgttgtggtaatc

(19) United States
(12) Patent Application Publication
(10) Pub. No.: US 2008/0010698 A1
(43) Pub. Date: Jan. 10, 2008

(54) Expansin Polypeptides

(76) Inventors: Simon McQueen Mason, York (GB); Louise Jones, York (GB)

Correspondence Address: Womble Carlyle Sandridge & Rice, PLLC;Attn: Patent Docketing 32nd Floor, P.O. Box 7037, Atlanta, GA, 30357-0037, US

(21) Appl. No.: 10/573,245

(22) PCT Filed: Sep. 23, 2004
(86) PCT No.: PCT/GB04/04058

§ 371 Date: Mar. 23, 2006

(30) Foreign Application Priority Data
Sep. 24, 2003 (GB) 0322317.9

(51) Int. Cl. C12N 15/82; A01H 5/00; C07H 21/04; C12N 5/04; C07K 14/00

(52) U.S. Cl. 800/278; 435/410; 530/350; 536/23.6; 800/298

(57) ABSTRACT

Transgenic plant cells and plants which have been genetically modified with nucleic acid molecules which encode expansin polypeptides: the disclosed are methods to alter the composition of plant cell walls, and products comprising plant tissue derived from said transgenic plants.

### CROSS REFERENCE TO RELATED APPLICATIONS

This application is a §371 National Stage application of International Application No. PCT/GB04/04058 filed Sep. 23, 2004 which claims priority to Great British Application No. 03223179 filed Sep. 24, 2003.

### BACKGROUND OF THE INVENTION

The invention relates to transgenic plant cells which have been genetically modified with nucleic acid molecules which encode expansin polypeptides; including methods to alter the composition of plant cell walls; and products, e.g. foods and paper, comprising plant tissue derived from said transgenic plants.

The plant cell wall forms the basis for many industrial and commercial products. Cellulose is the most abundant polymer found in nature and is used in the paper industry, and used as an anti-caking agent, emulsifier, stabilizer, dispersing agent and a thickening/gelling agent in foodstuffs. Cellulose also has excellent properties with respect to absorbing water which allows it to become soft and flexible.

The primary cell wall of a plant is a network of cellulose microfibrils embedded in a hemicellulose polysaccharide matrix which interacts with other molecular structural elements e.g. pectins. The cell wall is a structure which determines the cell shape and functions to stick cells to one another to form a structure which is largely impervious (e.g. to plant pathogens) and which has variable strength and rigidity. The polysaccharides of a growing plant cell wall are separate long chain polymers that form a network of non-covalent associations which confers a degree of structural flexibility on the cell wall. This flexibility allows a plant cell to withstand considerable mechanical stress that arises, for example, as a consequence of turgor pressure within the cell. This versatility primarily results from movement or rearrangement of matrix polymers. A class of cell wall proteins involved in this re-modelling are the expansins.

Plant cells originate from a small population of dividing cells referred to as meristems which are located at growth regions such as shoots and roots. Meristematic cells are very small being around 5 μm. Cells which become displaced from the meristem undergo a massive growth phase which results in an increase in cell volume which primarily results from the formation of a vacuole which functions as a sink for water and other solutes. The ability of the plant cell to undergo this transformation is in part due to the pliability of the cell wall. Expansins are intimately involved in this process.

Expansins are a group of proteins with a molecular mass of approximately 26 kDa and were first isolated from cucumber seedlings. Expansins exist as a large group of extracellular enzymes and have subsequently been identified in a wide range of plant species (Lee et al., 2001). They are now commonly divided into 2 major groups, referred to as \acute{α}- and β-expansins (Cosgrove et al., 2002), however, a third minor group of γ-expansins has also now been identified in Arabidopsis (Li et al., 2002). Most studies have focussed on the \acute{α}-expansins as they were the first to be characterized, they display a weak but significant sequence similarity to β-expansins, also referred to as the group I grass pollen allergens (Darley et al., 2001). The precise biochemical nature and role of these 2 groups of expansins remains to be fully elucidated however differing substrate specificities may well be a key factor.

The role of \acute{α}-expansins in cell growth through the mediation of pH-dependent cell wall extension has now been firmly established. The initial identification of expansin activity demonstrated their ability to restore pH-dependent wall extensibility to heat-inactivated cell walls (McQueen-Mason et al., 1992). More recently, a number of studies have verified correlations between growth and expression of expansins (Cho and Cosgrove, 2000; Im et al., 2000; Lee et al., 2003). The mechanism of expansin action is thought to involve the disruption of non-covalent bonds between cell wall polysaccharides, namely cellulose and xyloglucan (Whitney et al., 2000), allowing the polymers to slide apart and extension to occur. In addition to their role in growth, expansins have been shown to be involved in morphogenesis (Cleland, 2001; Pien et al., 2001; Reinhardt et al., 1998), germination (Chen and Bradford, 2000) and fruit softening (Brummell et al, 1999).

Expansin expression is also differentially regulated in terms of developmental, hormonal, and environmental factors (Cho and Cosgrove, 2002). One such environmental aspect affecting the differential expression of expansins is water availability. For example, in order to avoid drowning, semiaquatic plants are often induced to grow rapidly upon submergence in water as a means of survival. In both deepwater rice (Cho and Kende, 1997) and the flooding-tolerant Rumex palustris (Vriezen et al., 2000), submergence also results in an increase in expression of some expansin genes and this has been directly correlated with the observed increases in growth. In contrast, under conditions where water is limiting, roots adapted to environments where low water potentials prevail are able to continue growing by increasing the extensibility of their cell walls (Wu et al., 1996). This enhanced extensibility has been correlated with an increase in expansin activity and transcript accumulation (Wu et al., 2001).

Resurrection plants are a small but unique group of vascular plants that display an extreme level of tolerance to desiccation throughout their vegetative tissues (Scott, 2000). The resurrection plant Craterostigma plantagineum has served as a model species for the molecular investigations of desiccation tolerance (Ingram and Bartels, 1996; Ramanjulu and Bartels, 2002). Resurrection plants can typically survive losses of 95% of their original water content and then rehydrate completely with full physiological activity resumed within hours of watering.

Drought stress is generally detrimental to plant health as a result of adverse effects on metabolism, irreparable damage to membrane and protein structures and to subcellular organisation. Any such injuries must therefore either be protected against or repaired in resurrection plants. Examples of these mechanisms include an accumulation of sugars (such as sucrose) and protective proteins (eg. late embryogenesis-abundant (LEA) proteins), which are thought to stabilize the structural integrity of macromolecules during dehydration (Bartels and Salamini, 2001; Hoekstra et al., 2001). Protection of the photosynthetic apparatus by physical alterations in leaf shape (such as curling), anthocyanin biosynthesis and the reversible loss of chlorophyll can also occur (Alpert 2000).

A small number of resurrection plants, including Craterostigma plantagineum, also display extensive cell wall folding during dehydration (Hartung et al., 1998; Farrant 2000). This process of folding of the wall is thought to alleviate the tension, and potential damage, arising as a result of the plasma membrane contracting as the cell shrinks during dehydration whilst still attached to the wall via plasmodesmata. Maintaining close associations between the cell wall and plasma membrane would also be expected to be vital during rehydration and water uptake. A previous study has suggested that this mechanism of cell wall folding in Craterostigma is accompanied by changes in wall architecture and composition (Vicre et al., 1999).

The ability of some resurrection plants to alter the mechanical properties of their cell walls allowing an apparent increased extensibility and flexibility during dehydration and rehydration is a unique characteristic and vital to their survival.

### DESCRIPTION OF THE INVENTION

We have examined a potential role for expansins in relation to this aspect of desiccation tolerance in Craterostigma plantagineum. We describe the isolation of three expansin genes from C. plantagineum and demonstrates a clear correlation between the expression of expansin genes and the dehydration and rehydration events that enable desiccation tolerance.

According to an aspect of the invention there is provide a transgenic plant cell wherein said plant cell is genetically modified by transformation with a nucleic acid molecule selected from the group consisting of:
i) a nucleic acid molecule comprising a nucleic acid sequence as represented in FIG. 6;
ii) a nucleic acid molecule which hybridises to the nucleic acid molecule as defined in (i) and which encodes a polypeptide which has the specific activity associated with an expansin;
iii) a nucleic acid molecule that encodes an expansin characterized by the amino acid sequence motif ASSISGGG (SEQ ID NO: 7);
iv) a nucleic acid molecule comprising a nucleic acid sequence which is degenerate as a result of the genetic code to the sequences defined in (i) and (ii).

In a preferred embodiment of the invention said plant cell is adapted for the over-expression of said nucleic acid molecule.

In a preferred embodiment of the invention said nucleic acid molecule hybridizes under stringent hybridization conditions to a nucleic acid molecule as represented in FIG. 6a, b or c. Preferably said nucleic acid molecule consists of the nucleic acid sequence represented in FIG. 6a, b or c.

Stringent hybridization/washing conditions are well known in the art, for example, washing in 0.1×SSC, 0.1% SDS at 60° C. It is well known in the art that optimal hybridisation conditions can be calculated if the sequence of the nucleic acid is known. For example, hybridization conditions can be determined by the GC content of the nucleic acid subject to hybridization. Please see Sambrook et al (1989) Molecular Cloning; A Laboratory Approach. A common formula for calculating the stringency conditions required to achieve hybridization between nucleic acid molecules of a specified homology is:

Tₘ=81.5° C.+16.6 Log [Na⁺]+0.41[% G+C]−0.63 (% formamide).

Typically, hybridization conditions use 4−6×SSPE (20×SSPE contains 175.3 g NaCl, 88.2 g NaH₂PO₄ H₂O and 7.4 g EDTA dissolved to 1 liter and the pH adjusted to 7.4); 5-10× Denhardts solution (50× Denhardts solution contains 5 g Ficoll (type 400, Pharmacia), 5 g polyvinylpyrrolidone and 5 g bovine serum albumen; 100 μg-1.0 mg/ml sonicated salmon/herring DNA; 0.1-1.0% sodium dodecyl sulphate; optionally 40-60% deionised formamide. Hybridization temperature will vary depending on the GC content of the nucleic acid target sequence but will typically be between 42°-65° C.

In a preferred embodiment of the invention said nucleic acid molecule comprises a nucleic acid sequence which has at least 60% homology to the nucleic acid sequence represented in FIG. 6a, b, or c. Preferably said homology is at least 70%; 80%; 90%; or at least 99% identity with the nucleic acid sequence represented in FIG. 6a, b, or c.

In a further preferred embodiment of the invention said cell is transformed with a nucleic acid molecule which encodes a polypeptide as represented by the amino acid sequence in FIG. 7a, b or c or a variant polypeptide wherein said variant is modified by addition, deletion or substitution of at least one amino acid residue.

In a preferred embodiment of the invention said plant cell is transformed with a nucleic acid molecule which encodes an expansin polypeptide wherein said nucleic acid molecule is isolated from the genome of a resurrection plant. Preferably said resurrection plant is of the genus Craterostigma spp, preferably C. plantaagineum.

In a further preferred embodiment of the invention said nucleic acid molecule is over-expressed at least 2-fold when compared to basal level expression.

“Basal level expression” may be construed as the level of expression shown by a non-transgenic reference cell of the same species or a transgenic reference cell which contains a non-functional copy of the gene or cDNA of interest.

In a further preferred embodiment of the invention said cell over-expresses said nucleic acid molecule at least 5-fold, 10-fold, 20-fold, 30-fold, 40-fold, 50-fold. Preferably said cell over expresses said nucleic acid molecule at least 100-fold.

It will be apparent that means to increase the activity of a polypeptide encoded by a nucleic acid molecule are known to the skilled artisan. For example, and not by limitation, increasing the gene dosage by providing a cell with multiple copies of said gene. Alternatively or in addition, a gene(s) may be placed under the control of a powerful promoter sequence or an inducible promoter sequence to elevate expression of mRNA encoded by said gene. The modulation of mRNA stability is also a mechanism used to alter the steady state levels of an mRNA molecule, typically via alteration to the 5′ or 3′ untranslated regions of the mRNA.

In a further preferred embodiment of the invention said cell is transfected with a vector comprising a nucleic acid molecule selected from the group consisting of:
i) a nucleic acid molecule comprising a nucleic acid sequence as represented in FIG. 6a, b or c;
ii) a nucleic acid molecule which hybidises to the nucleic acid molecule as defined in (i) and which encodes a polypeptide which has the specific activity associated with an expansin;
iii) a nucleic acid molecule that encodes an expansin characterised by the amino acid sequence motif ASSISGGG (SEQ ID NO: 7);
iv) a nucleic acid molecule comprising a nucleic acid sequence which is degenerate as a result of the genetic code to the sequences defined in (i) and (ii).

Preferably the nucleic acid in the vector is operably linked to an appropriate promoter or other regulatory elements for transcription in a host cell such as a prokaryotic, (e.g. bacterial), or a plant cell. The vector may be a bi-functional expression vector which functions in multiple hosts. In the example of nucleic acids encoding polypeptides according to the invention this may contain its native promoter or other regulatory elements and in the case of cDNA this may be under the control of an appropriate promoter or other regulatory elements for expression in the host cell.

By “promoter” is meant a nucleotide sequence upstream from the transcriptional initiation site and which contains all the regulatory regions required for transcription. Suitable promoters include constitutive, tissue-specific, inducible, developmental or other promoters for expression in plant cells comprised in plants depending on design. Such promoters include viral, fungal, bacterial, animal and plant-derived promoters capable of functioning in plant cells.

Constitutive promoters include, for example CaMV 35S promoter (Odell et al (1985) Nature 313, 9810-812); rice actin (McElroy et al (1990) Plant Cell 2: 163-171); ubiquitin (Christian et al. (1989) Plant Mol. Biol. 18 (675-689); pEMU (Last et al (1991) Theor Appl. Genet. 81: 581-588); MAS (Velten et al (1984) EMBO J. 3. 2723-2730); ALS promoter (U.S. application Ser. No. 08/409,297), and the like. Other constitutive promoters include those in U.S. Pat. Nos. 5,608,149; 5,608,144; 5,604,121; 5,569,597; 5,466,785; 5,399,680, 5,268,463; and 5,608,142.

Chemical-regulated promoters can be used to modulate the expression of a gene in a plant through the application of an exogenous chemical regulator. Depending upon the objective, the promoter may be a chemical-inducible promoter, where application of the chemical induced gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al (1991) Proc. Natl. Acad. Sci. USA 88: 10421-10425 and McNellie et al. (1998) Plant J. 14(2): 247-257) and tetracycline-inducible and tetracycline-repressible promoters (see, for example, Gatz et al. (1991) Mol. Gen. Genet. 227: 229-237, and U.S. Pat. Nos. 5,814,618 and 5,789,156, herein incorporated by reference.

Where enhanced expression in particular tissues is desired, tissue-specific promoters can be utilised. Tissue-specific promoters include those described by Yamamoto et al. (1997) Plant J. 12(2): 255-265; Kawamata et al (1997) Plant Cell Physiol. 38(7): 792-803; Hansen et al (1997) Mol. Gen. Genet. 254(3): 337-343; Russell et al. (1997) Transgenic Res. 6(2): 157-168; Rinehart et al (1996) Plant Physiol. 112(3): 1331-1341; Van Camp et al (1996) Plant Physiol. 112(2): 525-535; Canevascni et al (1996) Plant Physiol. 112(2): 513-524; Yamamoto et al (1994) Plant Cell Physiol. 35(5): 773-778; Lam (1994) Results Probl. Cell Differ. 20: 181-196; Orozco et al (1993) Plant Mol. Biol. 23(6): 1129-1138; Mutsuoka et al (1993) Proc. Natl. Acad. Sci. USA 90(20): 9586-9590; and Guevara-Garcia et al (1993) Plant J. 4(3): 495-50.

“Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is an inducible promoter or a developmentally regulated promoter.

Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148. Suitable vectors may include plant viral-derived vectors (see e.g. EP-A-194809).

Vectors may also include selectable genetic marker such as those that confer selectable phenotypes such as resistance to herbicides (e.g. kanamycin, hygromycin, phosphinotricin, chlorsulfuron, methotrexate, gentamycin, spectinomycin, imidazolinones and glyphosate).

According to a further aspect of the invention there is provided a plant comprising a cell according to the invention.

In a preferred embodiment of the invention said plant is selected from: corn (Zea mays), canola (Brassica napus, Brassica rapa ssp.), flax (Linum usitatissimum), alfalfa (Medicago sativa), rice (Oryza sativa), rye (Secale cerale), sorghum (Sorghum bicolor, Sorghum vulgare), sunflower (Helianthus annus), wheat (Tritium aestivum), soybean (Glycine max), tobacco (Nicotiana tabacum), potato (Solanum tuberosum), peanuts (Arachis hypogaea), cotton (Gossypium hirsutum), sweet potato (Iopmoea batatus), cassava (Manihot esculenta), coffee (Cofea spp.), coconut (Cocos nucifera), pineapple (Anana comosus), citris tree (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, barley, vegetables and ornamentals.

Preferably, plants of the present invention are crop plants (for example, cereals and pulses, maize, wheat, potatoes, tapioca, rice, sorghum, millet, cassava, barley, pea), and other root, tuber or seed crops. Important seed crops are oil-seed rape, sugar beet, maize, sunflower, soybean, sorghum, and flax (linseed). Horticultural plants to which the present invention may be applied may include lettuce, endive, and vegetable brassicas including cabbage, broccoli, and cauliflower. The present invention may be applied in tobacco, cucurbits, carrot, strawberry, sunflower, tomato, pepper.

Grain plants that provide seeds of interest include oil-seed plants and leguminous plants. Seeds of interest include grain seeds, such as corn, wheat, barley, rice, sorghum, rye, etc. Oil seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava been, lentils, chickpea, etc.

According to a further aspect of the invention there is provided a product comprising a plant cell or plant tissue derived from a plant according to the invention.

In a preferred embodiment of the invention said product is a food stuff.

Many foods are stored and sold in a dried form, for example dried soups and fruits, which are rehydrated prior to use. The provision of a food stuff with improved rehydration properties which enhances the quality/texture of the food to make it more palatable has obvious benefits for the consumer.

In an alternative preferred embodiment of the invention said product is paper.

The recycling of paper presents particular problems. The production of pulp from paper which is to be recycled make use of chemical denaturants and enzymes. Although chemical methods are efficient means to remove, for example, lignin from pulp it is known that chemical treatments can result in degradation of polysaccharides and are expensive. Moreover, to remove residual lignin from pulp it is necessary to use strong bleaching agents which require removal before the pulp can be converted into paper. These agents are also damaging to the environment. Biological methods to remove lignin are known. There are however disadvantages associated with such methods. For example it is important to provide micro-organisms (eg bacteria and/or fungi) which only secrete ligninolytic enzymes which do not affect cellulose fibres. This method is also very time consuming (can take 3-4 weeks) and expensive due to the need to provide bioreactors. Biological treatment can also include pre-treatment of wood chips to make them more susceptible to further biological or chemical pulping.

The paper products derived from transgenic plants as hereindisclosed which contain cellulose with altered mechanical properties would facilitate lignin extraction and provide a higher quality paper product due to the fact that the use of chemical/biological agents is reduced.

According to a yet further aspect of the invention there is provided a method to alter the mechanical properties of a plant cell wall comprising the steps of:
i) providing a cell according to the invention; and
ii) cultivating from said cell a plant.

In a preferred method of the invention said plant, or part thereof, is dehydrated.

According to a further aspect of the invention there is provided a method to prepare a cell wall extract wherein said cell wall has altered mechanical properties comprising the steps of:
i) providing a cell according to the invention;
ii) cultivating said cell into a plant; and
iii) preparing a cell wall extract from said plant.

In a preferred method of the invention said extract is dehydrated.

According to a further aspect of the invention there is provided a cell wall extract prepared by the method according to the invention.

According to a yet further aspect of the invention there is provided a composition comprising a polypeptide encoded by a nucleic acid molecule selected from the group consisting of:
i) a nucleic acid molecule comprising a nucleic acid sequence as represented in FIG. 6a and/or b and/or c;
ii) a nucleic acid molecule which hybridises to the nucleic acid molecule as defined in (i) and which encodes a polypeptide which has the specific activity associated with an expansin;
iii) a nucleic acid molecule that encodes an expansin characterised by the amino acid sequence motif ASSISGGG;
iv) a nucleic acid molecule comprising a nucleic acid sequence which is degenerate as a result of the genetic code to the sequences defined in (i), (ii) or (iii).

In a preferred embodiment of the invention there is provided the use of a composition according to the invention as an agent for the rehydration of dehydrated plant material.

An embodiment of the invention will now be described by example only and with reference to the following figures:

### BRIEF DESCRIPTION OF THE DRAWINGS

FIG. 1. 0.5 μm toluidine blue stained sections of resin-embedded Craterostigma plantagineum leaf tissue following 0 hours dehydration (a,b) and 48 hours dehydration (c,d). mc, mesophyll cells; vc, vascular cells; xy, xylem. Areas demonstrating extensive cell wall folding are indicated (*).

FIG. 2. (a) Expansin activity in dehydrating and rehydrating Craterostigma plantagineum leaves. Crude cell wall protein extracts were isolated from Craterostigma leaf material dehydrated and then subsequently rehydrated for specific periods of time. Activity was measured according to an increase in elongation rate following the addition of expansins to a cellulose/xyloglucan composite under a constant load. Measurements are expressed relative to a standardized protein concentration. Data are averages and standard errors from 6 separate measurements. Overall experiments were repeated 3 times with similar results. (b) Images depicting the whole leaf morphology of detached C. plantagineum leaves at various stages of dehydration and rehydration. Time points are the same as those used for assaying expansin activity.

FIG. 3. Protein sequence alignment of \acute{α}-expansins from Craterostigma plantagineum (CplExp1, CplExp2 and CplExp3), Arabidopsis (AthExp\acute{α}d1.2, GenBank accession number AAG60095; AthExp\acute{α}1.5, GenBank accession number BAB11259; AthExp\acute{α}1.6, GenBank accession number AAB97125) and maize (ZmaExp2, GenBank accession number AAK56120). Shaded areas represent regions where sequences align, the color scheme denotes conserved residues (black) and similar residues (grey).

FIG. 4 illustrates expansin activity during dehydration and rehydration of detached Craterostigma plantagineum leaves.

FIG. 5. Expression of 3 \acute{α}-expansin genes from Craterostigma plantagineum at specific stages of dehydration and rehydration, as determined by Real-Time PCR. Transcript levels are expressed relative to a calibrator, or endogenous control, in this case the transketolase3 gene. The expression of this gene has been previously shown to be unaffected by dehydration or rehydration events;

FIGS. 6a, b and c are the nucleic acid sequence of Craterostigma plantagineum expansins;

FIGS. 7a, b and c is the amino acid sequence of Craterostigma plantagineum expansin; and

FIG. 8 illustrates the effect of expansins on rehydration of dried plant material.

### EXAMPLES

### Materials and Methods

### Plant Material

Craterostigma plantagineum plants were grown in growth rooms set at 20° C. with 16 hour day length. Dehydration and rehydration experiments were carried out on detached leaves from mature plants, approximately 10 weeks old, under growth room conditions.

### Microscopy

Untreated leaf tissue (approximately 5 mm² sections), 48 hours dehydrated leaf material and 24 hours rehydrated leaf tissue (following 48 hours dehydration) were vacuum infiltrated with 4% paraformaldehyde in 0.1 M phosphate buffer and incubated overnight in fixative. Leaf material was then washed in 0.1 M phospate buffer and dehydrated through a graded ethanol series and finally embedded in LR White resin (Agar Scientific). Semi-thin 0.5 μm sections were cut with a Leica Ultracut UCT and stained with 0.6% toluidine blue in 0.3% sodium carbonate.

### Expansin Activity Assays

Detached leaves were dehydrated for 0, 3, 8, 24 and 48 hours and then rehydrated for a further 3, 5 and 24 hours. Leaves were frozen in liquid nitrogen and stored at −80° C. 5 g of frozen leaf material was disrupted in a domestic food homogeniser (Philips Cucina) with a 150 ml cup by blending in 50 ml of homogenisation buffer (25 mM Hepes, 2 mM EDTA, 3 mM sodium metabisulfite, 3 mM dithiothreitol, pH 6.8) at full power for 1 min. Leaf fragments were captured on a 50 μm pore size nylon membrane and the liquid discarded. Fragments were then washed 3 times in 50 ml of the homogenisation buffer before being squeezed dry in the membrane. Washed leaf fragments were suspended in 10 ml of 1 M NaCl, 25 mM Hepes, 2 mM EDTA, 3 mM sodium metabisulfite, 3 mM dithiothreitol, pH 6.8 and left for 1 h with gentle rocking at room temperature. The extractant was passed through the nylon mesh and proteins were precipitated by slowly dissolving 0.39 g/ml solid ammonium sulfate in the clarified extract. Precipitates were collected by centrifugation at 10,000 g at 4° C. for 10 min, resuspended in 1 ml of 50 mM sodium acetate, pH 4.5 and desalted on a 5 ml column of Sephadex G25 (Amersham Pharmacia Biotech). Protein concentrations were calculated using the Coomassie Plus Protein Assay Reagent (Pierce) following the manufacturers instructions.

Protein extracts were assayed for expansin activity as described by Whitney et al. (2000). Briefly, 2 mm wide strips of cellulose/xyloglucan composite, produced by cultures of Acetobacter xylinus, were cut and held between the two clamps of a custom made extensometer leaving 3 mm of material between the clamps. The composite was bathed in 50 mM sodium acetate, pH 4.5 and allowed to extend for 15 min under an applied force of 5 g. After 15 min the bathing solution was replaced with one containing the desalted extracted proteins in the same buffering solution. Extension was monitored for a further 15 min. Expansin activity was calculated as the rate of extension of the composite in the 10 min period following protein addition minus the rate of extension in the 10 min period prior to protein addition.

### RT-PCR

Total RNA was extracted according to standard protocols from Craterostigma leaf tissue dehydrated for 3 hours. Reverse transcription (RT) was carried out with 1 μg total RNA, 500 ng Oligo(dT)₁₂₋₁₈ primer (Invitrogen) and 200 Units of Superscript II RNase H⁻ Reverse Transcriptase (Invitrogen) in a final volume of 20 μl for 1 hour at 42° C., in line with the manufacturers instructions. A control reaction was performed in the absence of reverse transcriptase.

PCR was carried out with the synthesized cDNA, Taq DNA polymerase (Invitrogen) and concensus expansin primers. The primer sequences used were 5′-GSNCAYGCNACNTTYTAYGGNG-3′ (forward primer) (SEQ ID NO: 8) and 5′-YTGCCARTTYTGNCCCCARTT-3′ (reverse primer) (SEQ ID NO: 9). PCR cycling parameters were as follows: 92° C. for 2 minutes, then 35 cycles of 92° C. for 30 seconds, 50° C. for 30 seconds, 72° C. for 1 minute, and finally 72° C. for 5 minutes.

The PCR products were cloned into pCR2.1-TOPO vector (Invitrogen) according to the manufacturers instructions and sequenced.

3′ and 5′ RACE

Full length coding sequence for CplExp1 was obtained by 3′ and 5′ rapid amplification of cDNA ends (RACE). For 3′ RACE, reverse transcription was carried out as described above using an adapter oligo(dT) 3′ primer (5′-GACCACGCGTATCGATGTCGACTTTTTTTTTTTTTTTTV-3′) (SEQ ID NO: 10). PCR was then performed using a gene specific 5′ primer (5′-GTCACAGCCTGTTTTCCAACAC-3′) (SEQ ID NO: 11), the adapter primer and Taq DNA polymerase (Invitrogen). A further PCR amplification was carried out using a nested 5′ gene specific primer (5′-ACTAACGTGGGCGGTGCT-3′) (SEQ ID NO: 12). The final product was then cloned into pCR2.1-TOPO and sequenced.

For 5′RACE, reverse transcription was performed with a 3′ gene specific primer (5′-GCCTCCTCTTCTACGGCATGGTACC-3′) (SEQ ID NO: 13) and a 5′adapter oligonucleotide (5′-AAGCAGTGGTATCAACGCAGAGTGGCCATTATGGCCGGG-3′) (SEQ ID NO: 14). PCR was carried out with the same 3′ gene specific primer and the 5′ adapter (5′-AAGCAGTGGTATCAACGCAGAGT-3′) (SEQ ID NO: 15). In order to improve specificity, a further round of PCR was performed with a nested 3′ gene specific primer (5′-CTGTAAGAAACGGGCACAATCCCAGC-3′) (SEQ ID NO: 16). Products were cloned and sequenced as before.

### Real Time PCR

Reverse transcription (RT) reactions were carried out as described above using 2 μg RNA extracted from Craterostigma leaf tissue dehydrated for 0, 1, 3, 8, 24 and 48 hours and then rehydrated for a further 1, 3, 8 and 24 hours. All RNA samples were DNase-treated prior to cDNA synthesis. Following RT, each reaction was purified using a Qiagen QIAquick PCR purification kit.

Real time PCR was carried out using relative quantitation based on the standard curve method such that quantities were expressed relative to an endogenous control, in this case tkt3 (transketolase3). Primer pairs were designed to each expansin sequence and the tkt3 gene using PrimerExpress (Applied Biosystems). Primer sequences and the optimised concentrations used in each of the PCR reactions were as follows: CplExp1 Forward (100 nM) 5′-GCTCAGTATACAGCTGGGATTGTG-3′ (SEQ ID NO: 17), CplExp1 Reverse (200 nM) 5′-TTGAAGTAAGAGTGTCCGTTTATTGTG-3′ (SEQ ID NO: 18), CplExp2 Forward (300 nM) 5′-GTGCCTCTCGGGAACCATAAT-3′ (SEQ ID NO: 19), CplExp2 Reverse (300 nM) 5′-TTGTACTGCGCTATCTGCAAGAA-3′ (SEQ ID NO: 20), CplExp3 Forward (300 nM) 5′-CGCTGAGTACAACGCTGTTCA-3′ (SEQ ID NO: 21), CplExp3 Reverse (50 nM) 5′-GTAATTGGGAGGACAGAAATTTGTG-3′ (SEQ ID NO: 22), tkt3 Forward (300 nM) 5′-CATCTGGGTTAAGAACGGAAACA-3′ (SEQ ID NO: 23) and tkt3 Reverse (50 nM) 5′-CAAAACCGATCGTTGTGGTAATC-3′ (SEQ ID NO: 24).

25 μl PCR reactions were carried out using the SYBR Green PCR master mix (Applied Biosystems) in optical 96-well reaction plates (Applied Biosystems) on an ABI Prism 7000 Sequence Detection System. Cycling parameters were as follows: 50° C. for 2 minutes, 95° C. for 10 minutes and then 40 cycles of 95° C. for 15 seconds, 60° C. for 1 minute. All reactions were performed in triplicate or more.

### Example 1

### Rphological Changes During Dehydration

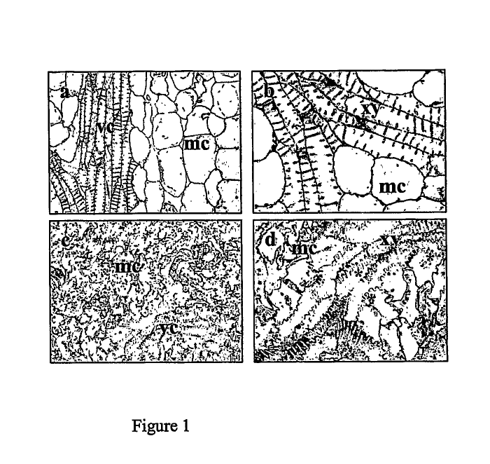

The dramatic alterations observed in the cellular structure of Craterostigma plantagineum leaves during dehydration is clearly demonstrated in FIG. 1. The regular appearance of the mesophyll and vascular cells in a typical hydrated leaf (FIG. 1a,b) was severely disrupted following 48 hours dehydration (FIG. 1c,d). As expected, water loss resulted in significant shrinkage of the mesophyll cells and was accompanied by extensive cell wall folding. The xylem vessels were also affected by dehydration but predominantly exhibited shrinkage in the longitudinal rather than tangential plane due to the presence of secondary cell wall thickenings characteristic of this cell type (FIG. 1d). The secondary thickenings alone appeared to maintain the vessels in an open state. However, cell wall folding was particularly prominent within the xylem and at junctions between vessels and mesophyll cells. Rehydration of leaf tissue following 48 hours dehydration resulted in complete recovery and a return to normal cellular morphology within the leaf.

### Example 2

### Expansin Activity During Dehydration and Rehydration

Expansin activity was assessed in crude cell wall protein extracts isolated from Craterostigma leaf material dehydrated for 0, 3, 8, 24 and 48 hours and then subsequently rehydrated for 3, 5 and 24 hours. Activity was measured according to an increase in elongation rate following the addition of proteins to a cellulose/xyloglucan composite under a constant load.

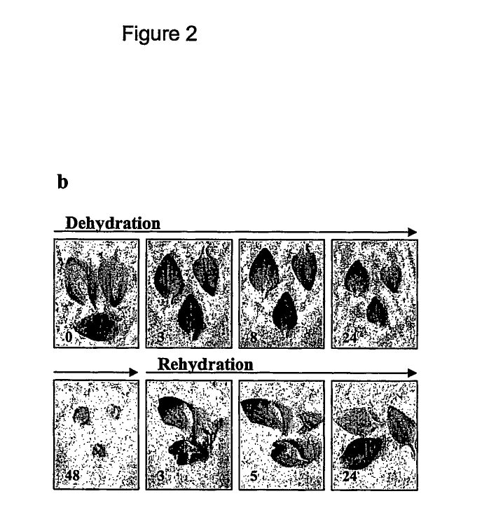

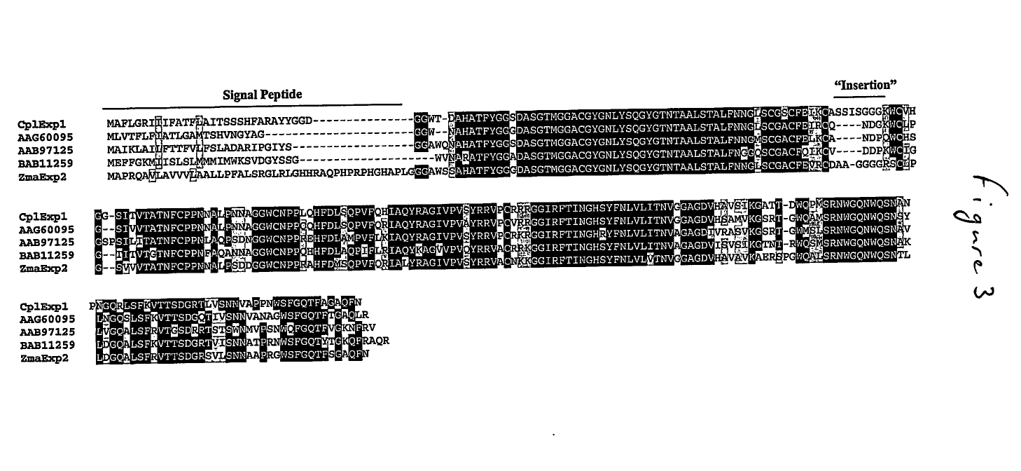

Results of expansin activity in detached Craterostigma leaves dehydrated and rehydrated for specific lengths of times indicated that activity varied significantly depending on the water content of the tissue (FIG. 2). Also shown in this figure are representative images of detached leaves at the same specified time points as those used to assay for expansin activity.

This data demonstrated that expansin activity increased steadily during the early periods of dehydration reaching a peak after 8 hours of an approximate 3-fold increase relative to the control hydrated leaves. Levels of activity then decreased throughout the latter stages of dehydration with activity reduced to levels similar to those detected in fresh, hydrated leaves by 24 hours. Following 48 hours without water, expansin activity was reduced to levels below those of the control. An examination of morphological changes of detached leaves during this period of dehydration revealed that the early stages of water loss, up to 8 hours, resulted in comparatively minor alterations in leaf shape, however, a reduction in size was apparent. After 24 hours without water, the leaves had shrunk in size significantly and by 48 hours, leaves were again smaller and had undergone extensive curling.

Expansin activity was also assessed in rehydrating leaves following 48 hours of dehydration (FIG. 2). These measurements demonstrated a pronounced increase in expansin activity during the first 5 hours of water uptake. After only 3 hours subsequent to the addition of water, activity levels had increased more than 4-fold relative to the hydrated leaves. The highest levels of activity, an increase of more than 5-fold compared to the control, were detected following 5 hours of rehydration. Activity then declined and returned to levels similar to those observed in fresh leaves after 24 hours of rehydration. Alterations in leaf shape during rehydration occurred rapidly with considerable unfolding taking place in the first 3 hours after the addition of water. This expansion and unfurling had increased further by 5 hours and after 24 hours of rehydration the leaves had returned to their original morphological state.

### Example 3

### Isolation of Expansin cDNAs from Craterostigma plantagineum

Expansin sequences were amplified by PCR, from cDNA derived from C. plantagineum RNA extracted from leaf material dehydrated for 3 hours, using consensus primers designed to the \acute{α}sub-group of Arabidopsis expansins. RT-PCR products were cloned and sequenced resulting in the identification of a number of expansin cDNA sequences. Further analysis indicated that these sequences fell into 3 separate groups demonstrating the presence of 3 distinct expansin cDNAs in dehydrating Craterostigma leaves. These three genes have been named CplExp1, CplExp2 and CplExp3, respectively.

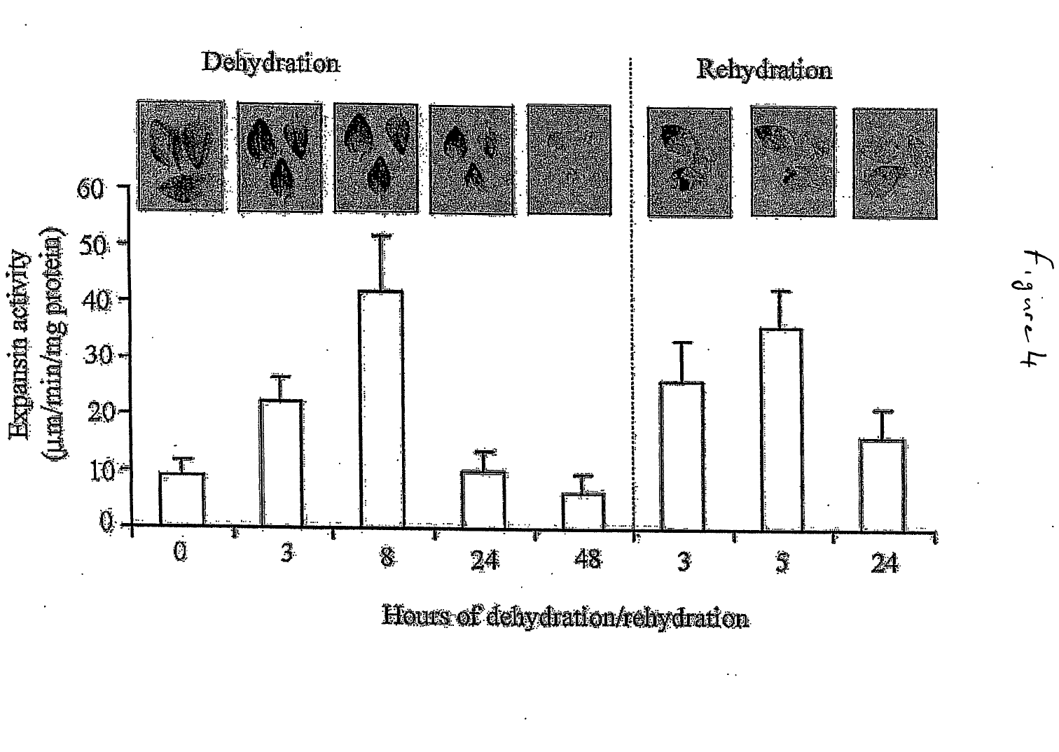

Full-length coding sequence was obtained for one of these genes, CplExp1, by 5′ and 3′ rapid amplification of cDNA ends revealing a protein composed of 261 amino acids possessing the characteristic signal peptide and motifs typical of the \acute{α}-expansins (FIG. 3). Only the 3′ ends, in addition to the sequence amplified with the consensus primers, were obtained for CplExp2 (216 amino acids) and CplExp3 (224 amino acids). Because of this, these sequences lack the signal peptide at the 5′ end however all other distinguishing \acute{α}-expansin motifs are present. At the nucleotide level, the 3 Craterostigma \acute{α}-expansins share approximately 70% sequence identity.

An alignment of these sequences together with 3 representative \acute{α}-expansins from Arabidopsis (AthExp\acute{α}1.2, GenBank accession number AAG60095; AthExp\acute{α}1.5, GenBank accession number BAB11259 and AthExp\acute{α}1.6, GenBank accession number AAB97125) and 1 from maize (ZmaExp2, GenBank accession number AAK56120) is shown in FIG. 3. This data clearly demonstrates the significant degree of homology between the C. plantagineum expansins isolated here and other previously defined \acute{α}-expansins. As previously noted, a large degree of heterogeneity exists at the 5′ end of these sequences which encodes the signal peptide for these proteins. All other domains of these \acute{α}-expansins are highly conserved. One notable exception resides in a short region between two highly conserved cysteines where CplExp1 exhibits a short 3 or 4 amino acid insertion (indicated by an asterisx above the sequence in FIG. 3). In a typical \acute{α}-expansin this region is approximately 6 amino acids long and is generally characterised by a series of 2-5 charged aminoacyl residues and a conserved tryptophan. In CplExp1 however, this stretch of protein sequence is 10 amino acids long and is predominantly composed of non-charged amino acids (CASSISGGGKWC). This region is not present in either CplExp2 or CplExp3 and sequence alignments indicate that only one other known expansin sequence, that of expansin 2 from maize, contains a remotely similar motif at this position.

Phylogenetic analysis of the Craterostigma \acute{α}-expansins together with those of Arabidopsis and rice demonstrated that all 3 sequences fell into sub-families and subgroups previously defined from the Arabidopsis and rice expansin family. Of the 3 protein sequences, only CplExp2 appeared to have a direct orthologue in either of the species, AthExp\acute{α}1.11. CplExp3 was more closely related to predominantly rice \acute{α}-expansins whilst CplExp1 was grouped with mostly Arabidopsis \acute{α}-expansins. It is also worth noting that CplExp1 resides within a distinct subgroup compared to CplExp2 and CplExp3.

### Example 4

### Expression of Expansins During Dehydration and Rehydration

RNA was isolated from detached C. plantagineum leaves at specific time points in order to assess the abundance of each of the 3 \acute{α}-expansin transcripts identified throughout the processes of dehydration and rehydration. Real-Time PCR was carried out using specific primers designed to each expansin and to the transketolase3 (tkt3) gene. Relative quantitation was carried out by constructing standard curves for each primer pair and quantities were expressed relative to an endogenous control. The tkt3 gene (GenBank accession number Z46646) was used as the endogenous control as this gene has previously been shown to be constitutively expressed and unaffected by dehydration or rehydration events (Bemacchia et al., 1995). Transcript levels of each expansin throughout dehydration and rehydration are presented in relation to hydrated (0 hour dehydration) tissue.

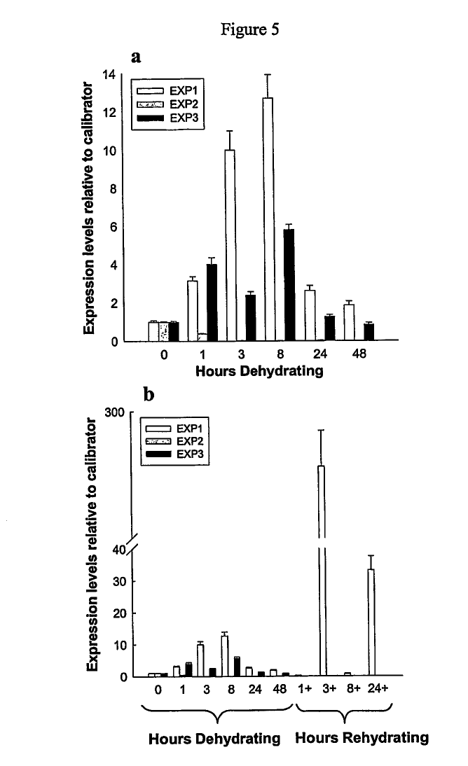

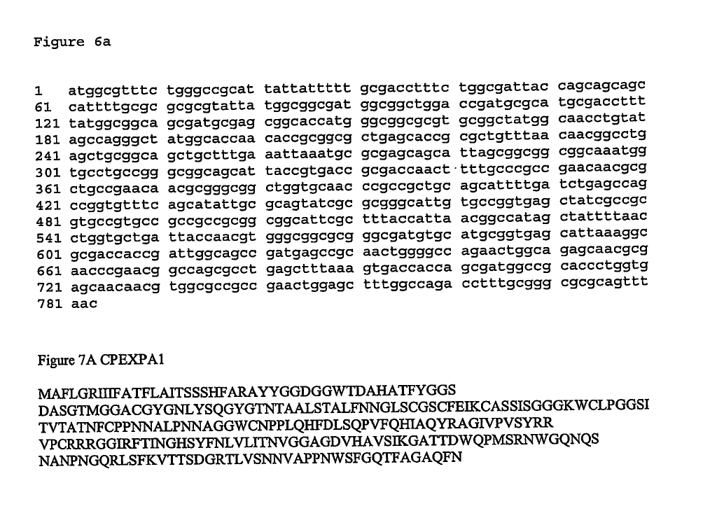

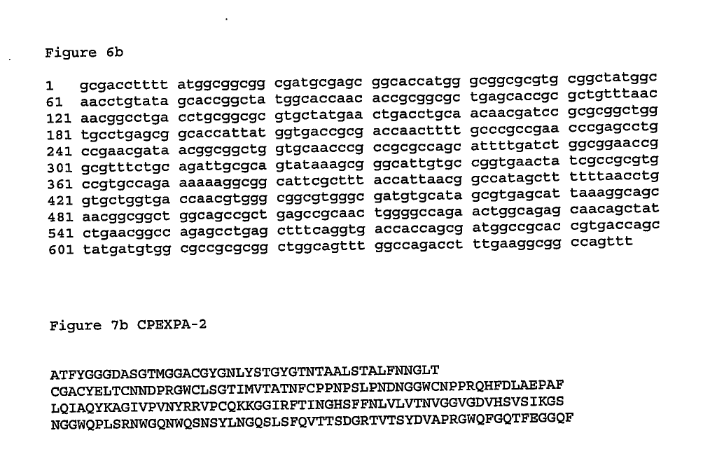

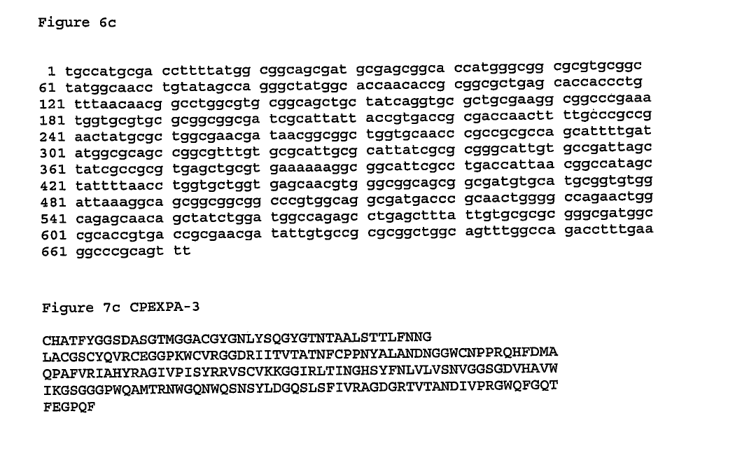

Data from Real-Time PCR experiments are shown in FIG. 5 and reveal that 2 (CplExp1 and CplExp3) of the 3 expansins underwent significant changes in expression throughout the 48 hour period of dehydration. Transcripts for CplExp1 and, to a lesser extent, CplExp3 increased in abundance relative to tkt3 during the early stages of dehydration with both peaking at 8 hours (FIG. 5a). Levels then declined steadily until the final time point of 48 hours without water. Throughout the 24 hours of rehydration, expression of CplExp3 remained largely unchanged whilst CplExp1 increased massively, reaching a peak of almost 300-fold relative to freshly harvested leaves, following 3 hours of rehydration (FIG. 5b). Transcript levels for CplExp1 had decreased sharply by the subsequent time point of 8 hours rehydrating before once again increasing around 30-fold after 24 hours of rehydration. In contrast, CplExp2 expression levels were largely unaffected by either dehydration or rehydration events.

The resurrection plant Craterostigma plantagineum undergoes extensive but reversible cell wall folding during dehydration of its vegetative tissues. In detached leaves dehydrated for 48 hours, morphological changes are significant with a considerable decrease in leaf size accompanied by leaf curling. At the cellular level, cell shrinkage and wall folding was evident throughout both the mesophyll and vascular cells. Wall folding was particularly prominent within the xylem, however, the collapse of these cells was prevented by the presence of secondary thickenings. This mechanism of wall folding is likely to alleviate some of the stresses incurred as cells shrink during dehydration and then expand throughout rehydration. As such, it is probable that these events play a significant role in the ability of C. plantagineum to survive desiccation.

Whilst this mechanism of wall folding is rare amongst resurrection plants it is not exclusive to Craterostigma plantagineum and suggests an uncommon degree of cell wall flexibility. Whether this is due to a unique cell wall composition, structural alterations in cell wall architecture during dehydration and rehydration, or is a dynamic process involving the synthesis of new wall components and the turnover of others remains to be established. However, a previous study has suggested that the mechanism of cell wall folding in Craterostigma wilmsii is accompanied by changes in wall architecture and composition (Vicre et al., 1999). These alterations indicated an increase in xyloglucan and unesterified pectins in the cell wall during dehydration with levels declining during rehydration.

We have examined a potential role for expansins in this process of cell wall folding during the dehydration and rehydration of Craterostigma plantagineum leaves. Experiments were carried out on detached leaves since they are capable of surviving desiccation and resuming physiological activity following rehydration and are therefore generally accepted as an accurate representation of whole plant physiology (Bartels et al., 1990). Assays for expansin activity indicated that activity varied significantly depending on the water content of the tissue with increases in activity occurring during the early stages of both dehydration and rehydration. The subsequent isolation of 3 \acute{α}-expansins from C. plantagineum enabled the analysis of alterations in transcript levels for each of these genes throughout the processes of dehydration and rehydration by Real-Time PCR. One of these genes, CplExp1, underwent significant changes in expression levels during the early stages of dehydration and throughout rehydration. CplExp3 demonstrated minor alterations in transcript levels during the preliminary stages of dehydration only whilst the expression of CplExp2 was largely unaffected. Taken together, these sets of data suggest a definite correlation between expansin activity and expression and the processes of dehydration and rehydration. One notable exception in the relationship between expansin activity and expression is the obvious disparity in the increase in transcript levels for CplExp1 during the early stages of rehydration and the corresponding increase in expansin activity. This may reflect a difference in the sensitivity of these alternative approaches. However, a more probable explanation may be that the adverse effects on metabolism caused by drought stress critically impair translation events during recovery.

Expansins have been shown to be involved in a number of developmental processes where the action of these enzymes permits wall extension, during growth for example, or wall breakdown or softening, such as during fruit ripening. As such, it is not difficult to envisage a role for expansins in the modification of the cell wall in Craterostigma during dehydration and rehydration. This may be a dynamic role involving the removal of wall components during dehydration and the synthesis of new polymers as the cells rehydrate and expand. This would combine both aspects of wall breakdown and wall extension. Alternatively, the role of expansins in desiccation tolerance may be by disrupting bonds between potentially the cellulose/xyloglucan network. This would therefore affect the structural integrity of the cell wall and may increase the flexibility by allowing polymers within the wall greater freedom of movement.

Previous studies have identified a number of dehydration-inducible (Neale et al., 2000; Kirch et al., 2001) and rehydration-inducible (Oono et al., 2003) genes, some of which have been cell wall-related, and have provided excellent candidates to improve desiccation tolerance and stress protection in other species. Sunkar et al. (2003) have shown that overexpression of an aldehyde dehydrogenase gene in Arabidopsis, identified in C. plantagineum as dehydration-inducible, improved tolerance to dehydration, salt, heavy metals, paraquat and hydrogen peroxide. Although no obvious orthologues exist for CplExp1 or CplExp3 in Arabidopsis or rice, the small insertion unique to CplExp1 composed predominantly of non-charged amino acids is an interesting motif requiring further investigation.

### Example 5

### Effects of Expansin Treatment on the Dehydration and Rehydration of Food Products

It is recognized that one of the major factors that interferes with the rehydration of dried fruit and vegetable products is the hardening of the cell wall and this is likely to result in part from the difficulty of rehydrating the extensively hydrogen-bonded polymer systems of the wall. Rehydration therefore depends in large measure on the plasticizing effects of water. Expansins are believed to disrupt hydrogen bonding between wall polymers and essentially function as wall plasticizers. Given the apparent role of expansins in the dehydration response in C. plantagineum, and their plasticizing effects on cell walls, these proteins may be useful in enhancing the ability of plant materials to dehydrate and rehydrate efficiently.

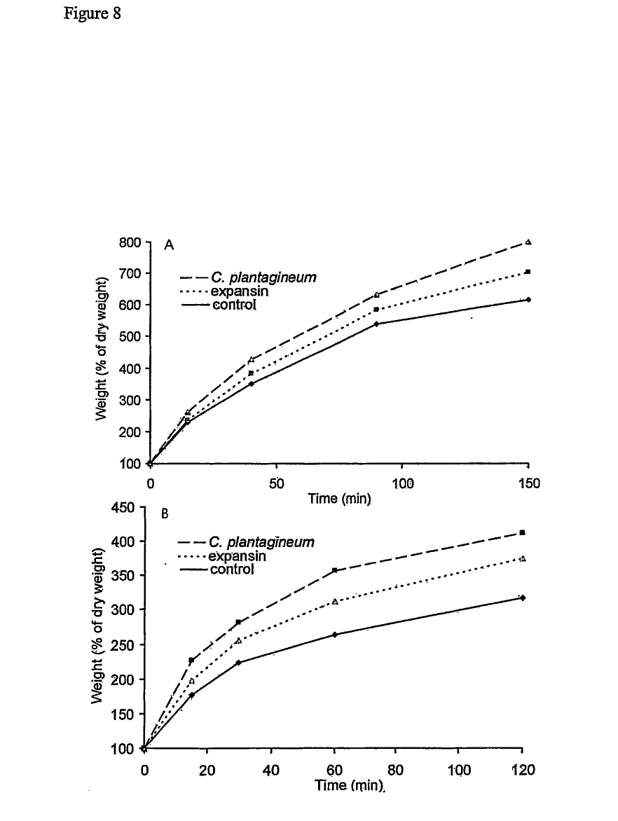

We have conducted initial experiments to examine the utility of expansins and protein extracts from C. plantagineum in rehydration assays and example results from these trials are presented in FIG. 8. We found that both recombinant cucumber expansin and C. plantagineum cell wall proteins effectively increased the uptake of water by dehydrated cylinders of both potato and carrot. These effects strongly support our hypothesis that insights gained from studying resurrection plants may have utility in industrial applications particularly in the area of dried foods. Interestingly, the cell wall extract from C. plantagineum appeared more effective than the recombinant cucumber expansin as in this crude extract. It is likely that the C. plantagineum expansin accounts for a only a small fraction of this crude extract and this may suggest that either the C. plantagineum expansin is more effective in these assays than the cucumber expansin.

### REFERENCES

Alpert P (2000) The discovery, scope, and puzzle of desiccation tolerance in plants. Plant Ecol 151: 5-17
Bartels D, Schneider K, Terstappen G, Piatkowski D, Salamini F (1990) Molecular cloning of abscisic acid-modulated genes which are induced during desiccation of the resurrection plant Craterostigma plantagineum. Planta 181: 27-34
Bartels D, Salamini F (2001) Desiccation tolerance in the resurrection plant Craterostigma plantagineum. A contribution to the study of drought tolerance at the molecular level. Plant Physiol 127: 1346-1353
Bernacchia G, Schwall G, Lottspeich F, Salamini F, Bartels D (1995) The transketolase gene family of the resurrection plant Craterostigma plantagineum: differential expression during the rehydration phase. EMBO J 14: 610-618
Brummell D A, Harpster M H, Civello P M, Palys J M, Bennett A B, Dunsmuir P (1999) Modification of expansin protein abundance in tomato fruit alters softening and cell wall polymer metabolism during ripening. Plant Cell 11: 2203-2216
Chen F, Bradford K J (2000) Expression of an expansin is associated with endosperm weakening during tomato seed germination. Plant Physiol 124: 1265-1274
Cho H T, Kende H (1997) Expansins and internodal growth of deepwater rice. Plant Physiol 113: 1145-1151
Cho H T, Cosgrove D J (2000) Altered expression of expansin modulates leaf growth and pedicel abscission in Arabidopsis thaliana. Proc Natl Acad Sci USA 97: 9783-9788
Cho H T, Cosgrove D J (2002) Regulation of root hair initiation and expansin gene expression in Arabidopsis. Plant Cell 14: 3237-3253
Cosgrove D J, Li L C, Cho H T, Hoffmann-Benning S, Moore R C, Blecker D (2002) The growing world of expansins. Plant Cell Physiol 43: 1436-1444
Darley C P, Forrester A M, McQueen-Mason S J (2001) The molecular basis of plant cell wall extension. Plant Mol Biol 47: 179-195
Farrant J M (2000) A comparison of mechanisms of desiccation tolerance among three angiosperm resurrection plant species. Plant Ecol 151: 29-39
Fleming A J, McQueenMason S, Mandel T, Kuhlemeier C (1997) Induction of leaf primordia by the cell wall protein expansin. Science 276: 1415-1418
Hartung W, Schiller P, Dietz K J (1998) Physiology of poikilohydric plants. In U Lüttge, ed, Progress in Botany 59. Springer-Verlag, Berlin, Heidelberg, pp 299-327
Hoekstra F A, Golovina E A, Buitink J (2001) Mechanisms of plant desiccation tolerance. Trends Plant Sci 6: 431-438
Im K H, Cosgrove D J, Jones A M (2000) Subcellular localization of expansin mRNA in xylem cells. Plant Physiol 123: 463-470
Ingram J, Bartels D (1996) The molecular basis of dehydration tolerance in plants. Ann Rev Plant Physiol Plant Mol Biol 47: 377-403
Kirch H H, Nair A, Bartels D (2001) Novel ABA- and dehydration-inducible aldehyde dehydrogenase genes isolated from the resurrection plant Craterostigma plantagineum and Arabidopsis thaliana. Plant J 28: 555-567
Lee D-K, Ahn J H, Song S-K, Choi Y D, Lee J S (2003) Expression of an expansin gene is correlated with root elongation in soybean. Plant Physiol 131: 985-977
Lee Y, Choi D, Kende H (2001) Expansins: ever-expanding numbers and functions. Curr Op Plant Biol 4: 527-532
Li Y, Darley C P, Ongaro V, Fleming A, Schipper O, Baldauf S L, McQueen-Mason S J (2002) Plant expansins are a complex multigene family with an ancient evolutionary origin. Plant Physiol 128: 854-864
Li Y, Jones L, McQueen-Mason S J (2003) Expansins and cell growth. Current Opinions Plant Biol
Maryani M M, Bradley G, Cahill D M, Gehring C A (2001) Natriuretic peptides and immunoreactants modify osmoticum-dependent volume changes in Solanum tuberosum L. mesophyll cell protoplasts. Plant Sci 61: 443-452
McQueen-Mason S J, Durachko D M, Cosgrove D J (1992) Two endogenous proteins that induce cell-wall extension in plants. Plant Cell 4: 1425-1433
Neale A D, Blomstedt C K, Bronson P, Le T N, Guthridge K, Evans J, Gaff D F, Hamill J D (2000) The isolation of genes from the resurrection grass Sporobolous stapfianus which are induced during severe stress. Plant, Cell Environ 23: 265-277
Oono Y, Seki M, Nanjo T, Narusaka M, Fujita M, Satoh M, Satou M, Sakurai T, Ishida J, Akiyama K, Iida K, Maruyama K, Satoh S, Yamaguchi-Shinozaki K, Shinozaki K (2003) Monitoring expression profiles of Arabidopsis gene expression during rehydration process after dehydration using ca. 7000 full-length cDNA microarray. Plant J 34: 868-887
Pien S, Wyrzykowska J, McQueen-Mason S, Smart C, Fleming A (2001) Local expression of expansin induces the entire process of leaf development and modifies leaf shape. Proc Natl Acad Sci USA 98: 11812-11817
Ramanjulu S, Bartels D (2002) Drought- and desiccation-induced modulation of gene expression in plants. Plant Cell Environ 25: 141-151
Reinhardt D, Wittwer F, Mandel T, Kuhlemeier C (1998) Localised up-regulation of a new expansin gene predicts the site of leaf formation in the tomato meristem. Plant Cell 10: 1427-1437
Rose J K C, Lee H H, Bennett A B (1997) Expression of a divergent expansin gene is fruit-specific and ripening-regulated. Proc Natl Acad Sci USA 94: 5955-5960
Scott P (2000) Resurrection plants and the secrets of eternal leaf. Annals Bot 85: 159-166
Sunkar R, Bartels D, Kirch H H (2003) Overexpression of a stress-inducible aldehyde dehydrogenase gene from Arabidopsis thaliana in transgenic plants improves stress tolerance. Plant J 35: 452-464
Vicre M, Sherwin H W, Driouch A, Jaffer M A, Farrant J M (1999) Cell wall characteristics and structure of hydrated and dry leaves of the resurrection plant Craterostigma wilmsii, a microscopical study. J Plant Physiol 155: 719-726
Vriezen W H, De Graaf B, Mariana C, Voesenek L A C J (2000) Submergence induces expansin gene expression in flooding-tolerant Rumex palustris and not in flooding-intolerant R. acetosa. Planta 210: 956-963
Whitney S E C, Gidley M J, McQueen-Mason S J (2000) Probing expansin action using cellulose/hemicellulose composites. Plant J 22: 327-334
Wu Y, Sharp R E, Durachko D M, Cosgrove D J (1996) Growth maintenance of the maize primary root at low water potentials involves increases in cell-wall extension proterties, expansin activity and wall susceptibility to expansins. Plant Physiol 111: 765-772
Wu Y, Thome E T, Sharp R E, Cosgrove D J (2001) Modification of expansin transcript levels in the maize primary root at low water potentials. Plant Physiol 126: 1471-1479

1-22. (canceled)

23. A transgenic plant cell wherein said plant cell is genetically modified by transformation or transfection with a nucleic acid molecule that encodes a plant expansin polypeptide comprising an amino acid sequence motif ASSISGGG.

24. The transgenic plant cell according to claim 23, wherein the nucleic acid molecule encodes an amino acid sequence selected from the group consisting of SEQ ID NOs: 2, 4, and 6.

25. The transgenic plant cell according to claim 24, wherein the nucleic acid molecule comprises a sequence selected from the group consisting of SEQ ID NOs: 1, 3 and 5.

26. The transgenic plant cell according to claim 25, wherein the nucleic acid molecule consists of a sequence selected from the group consisting of SEQ ID NOs: 1, 3 and 5.

27. The transgenic plant cell according to claim 23, wherein the nucleic acid molecule remains hybridized under a washing condition of 0.1×SSC, 0.1% SDS at 60° C. with a nucleic acid molecule consisting of a sequence selected from the group consisting of SEQ ID NOs: 1, 3 and 5.

28. The transgenic plant cell according to claim 23, wherein the cell over-expresses the nucleic acid molecule.

29. A cell according to any of claims 28, wherein said nucleic acid molecule is over-expressed at least 2-fold when compared to basal level expression.

30. A cell according to any of claims 28, wherein said cell over-expresses said nucleic acid molecule at least 100-fold.

31. The transgenic plant cell according to claim 23, wherein the nucleic acid molecule encodes a polypeptide that is at least 90% identical to an amino acid sequence selected from the group consisting of SEQ ID NOs: 2, 4, and 6.

32. The transgenic plant cell according to claim 31, wherein the nucleic acid molecule encodes a polypeptide that is at least 99% identical to an amino acid sequence selected from the group consisting of SEQ ID NOs: 2, 4, and 6.

33. The transgenic plant cell according to claim 23, wherein the nucleic acid molecule is isolated from the genome of a resurrection plant.

34. The transgenic plant cell according to claim 33, wherein the resurrection plant is a Craterostigma sp.

35. A plant comprising a cell according to claim 23.

36. A product comprising a plant cell or plant tissue derived from a plant according to claim 35.

37. A product according to claim 33 wherein said product is a food stuff, or paper.

38. A method for altering mechanical properties of a plant cell wall, the method comprising the steps of:
i) providing a plant cell according to claim 23, and
ii) cultivating from said cell a plant.

39. A method for preparing a cell wall extract wherein said cell wall has altered mechanical properties, the method comprising the steps of:
i) providing a plant according to claim 38; and
ii) preparing a cell wall extract from said plant.

40. A cell wall extract prepared by the method according to claim 39.

41. An isolated polypeptide having an amino acid sequence selected from the group consisting of SEQ ID NOs: 2, 4, and 6.

42. A method for the rehydration of dehydrated plant material, the method comprising applying a composition comprising a polypeptide according to claim 41.

43. An isolated polynucleotide molecule which encodes a polypeptide having an amino acid sequence selected from the group consisting of SEQ ID NOs: 2, 4, and 6.

44. The isolated polynucleotide molecule according to claim 43, wherein the polynucleotide comprises a nucleotide sequence of SEQ ID NO: 1, 3, or 5.
